(19) United States
(12) Patent Application Publication
(10) Pub. No.: US 2013/0078206 A1
(43) Pub. Date: Mar. 28, 2013

(54) Capsule of salts for pedicure bath

(71) Applicant: Viktor Dubinchuk, Tel Aviv (IL)

(72) Inventor: Viktor Dubinchuk, Tel Aviv (IL)

(21) Appl. No.: 13/200,904

(22) Filed: Sep. 28, 2011

(51) Int. Cl. A61K 8/96; B29C 39/02; A61Q 3/00

(52) U.S. Cl. 424/61; 264/328.1

(57) ABSTRACT

A water-soluble capsule for use in a pedicure bath that comprises the following substances: Dead Sea salts, olive oil, sodium bicarbonate, citric salt, starch, and fragrance. A method of manufacturing the capsule that includes the following stages: The aforementioned substances are placed in a mixer and a little water is added until a pourable mixture is obtained; the mixture is poured into a mold; pressure is applied to the capsule while in the mold; the mixture is dried in the mold until a solid capsule is obtained.

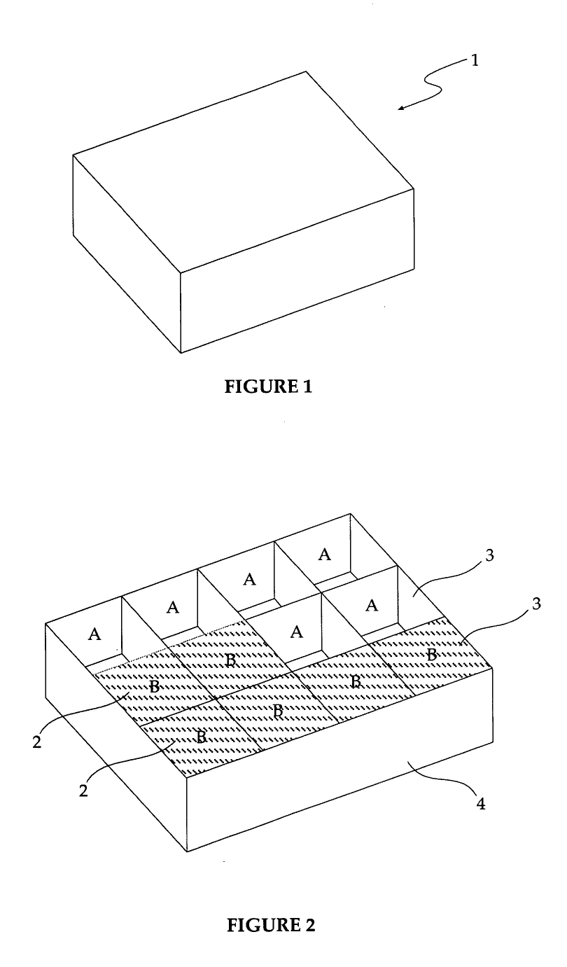

### TECHNICAL FIELD

The present invention refers to capsules of salt for use in pedicure baths and to a method for the manufacture of such.

### BACKGROUND ART

It is customary, at the beginning of a pedicure, to soak one's feet in a small foot bath (hereinafter the “bath”) that contains hot water, bath salts and occasionally bath oils and other substances. It is also customary for cosmeticians to have containers, tubes and bottles (hereinafter “containers”) of the said substances, which the cosmetician pours into the bath according to need. The need to have several such containers, to open and close them, and the dosage inaccuracy when pouring their contents each time a bath is filled, constitute a problem for the cosmetician. In addition, it is impractical for women who wish to give themselves a home-manicure to use such containers, since that would require having a stock of containers of the aforementioned substances at home. The present invention offers a solution to these problems by providing a water- soluble capsule that contains the said substances as well as fragrances for a pedicure bath. Thus, women can purchase a relatively small package containing several capsules for home use and simply add one capsule to the bath water each time they give themselves a pedicure. The capsule containing the said substances dissolves in the water and the bath is ready for use. Naturally, cosmeticians can also purchase such capsules instead of keeping a stock of containers on hand.

### THE INVENTION

The main objective of the present invention is to provide a water-soluble capsule that comprises pedicure salts, fragrance substances and other substances that help the compound the salts into the dissolvable solid state of the capsule. Another objective of the present invention is to specify the composition of the capsule and to describe the method used to manufacture it.

The capsule (1), subject of the present invention, contains the following substances: Dead Sea salts, olive oil, sodium bicarbonate, citric salt, starch and fragrances, such as lavender, vanilla, musk, peppermint, etc. The capsule may also contain other substances suitable for use in pedicure baths.

The recommended quantities needed to manufacture about 1 kg. capsule mixture are as follows: 700 g. Dead Sea salts, 150 g. olive oil, 50 g. sodium bicarbonate, 50 g. citric salt, 50 g. starch, and relatively small quantities of fragrance materials according to the type of material, type of fragrance, and desired fragrance intensity. Naturally, deviations from this recipe are possible and are also covered by the present patent application.

The present invention refers also to the method of manufacturing the capsule (1), as follows: All of the aforementioned substances are placed in a mixer and a small amount of water is added to form a pourable mixture (2). The mixture (2) is poured into a mold (3) as per the desired size of the capsule (1). The recommended dimensions of the capsule (1) are: length—3.5 cm., width—2 cm., and height—2 cm., although the patent application refers also to other capsule dimensions and shapes.

A tray (4) with several molds (3) can and should be used so as to make the manufacturing process of the capsule (1) more efficient. After pouring the mixture (2) into the mold (3), the tray (4) is vibrated so as to slightly compact the mixture (2). Then, pressure (weight) is applied to the tray to compact and compound the mixture (2). The mixture (2) is dried in the molds (3) and, when dry, the capsules (1) are released from the molds (3) and are ready for use. The mold (3) can and should have small perforation to enable liquid to drain from the mixture (2) during the drying process.

Drawing No. 1 depicts a block-shaped capsule (1).

Drawing No. 2 depicts a tray 4) with twelve molds (3), some of which are empty and are denoted A, and others that contain mixture (2) and are denoted B.

Claims what is claimed is:

1. A water-soluble capsule for use in a pedicure bath that comprises the following substances: Dead Sea salts, olive oil, sodium bicarbonate, citric salt, starch, and fragrance.

2. The capsule mentioned in claim No. 1 whereby its composition is as follows: 700 g. Dead Sea salts, 150 g. olive oil, 50 g. sodium bicarbonate, 50 g. citric salt, 50 g. starch, and fragrance.

3. A method for manufacturing the capsule mentioned in claim No. 1 that comprises the following stages: (1) The substances mentioned in claim No. 2 are placed in a mixer and a small amount of water is added until a pourable mixture is obtained; (2) the mixture is poured into the mold; (3) pressure is applied to the mixture while in the mold; and (4) the mixture is dried in the mold until a solid capsule is obtained.
